(19) United States
(12) Patent Application Publication
(10) Pub. No.: US 2019/0133470 A1
(43) Pub. Date: May 9, 2019

(54) SKIN TISSUE SENSOR DEVICE

(71) Applicant: iBeat, Inc., San Francisco, CA (US)

(72) Inventor: Steven Szabados, San Francisco, CA (US)

(21) Appl. No.: 15/967,956

(22) Filed: May 1, 2018

Related U.S. Application Data
(60) Provisional application No. 62/583,312, filed on Nov. 8, 2017.

(51) Int. Cl. A61B 5/024; A61B 5/0265; A61B 5/02; A61B 5/00; A61B 5/11

(52) U.S. Cl. CPC A61B 5/02427; A61B 5/02438; A61B 5/02405; A61B 5/0265; A61B 5/02028; A61B 5/7282; A61B 2560/0223; A61B 5/747; A61B 5/02433; A61B 5/6844; A61B 2562/0238; A61B 2562/0271; A61B 2560/0252; A61B 5/1113

(57) ABSTRACT

A dermal and cardiovascular spectroscopic sensor includes a number of light-emitting diodes (LEDs) having strategic locations and frequencies, and photo-detectors matched by wavelength of detection for measuring reflections of visible and infra-red light emitted by the LEDs. Spatially resolved spectroscopy is used to determine vital signs of the individual, including pulse sensing, tissue hemodynamics testing, and blood profusion testing. Capacitive and skin contact pads are used for determining that the sensor is being worn and is properly positioned. An accelerometer can be used to determine an activity level for the individual and if a physical mishap has occurred. A temperature sensor can be used to calibrate the digital monitoring device.

### RELATED U.S. APPLICATION

This application claims priority to U.S. Provisional Application No. 62/583,312, entitled “Dermal and Cardiovascular Spectroscopic Sensor,” filed on Nov. 8, 2017, which is hereby incorporated by reference in its entirety.

### TECHNICAL FIELD

Embodiments according to the present invention relate to the field of wearable health devices, and more particularly to a sensor technology used with a wearable health monitoring system.

### BACKGROUND

Health monitoring devices have become available in a wearable format, such as those that are wearable on a user's wrist. Many of the devices use sensors that contact a user's skin.

There is a need in the art for improved sensors, for accurate detection and reporting of anomalies relating to cardiovascular activity or tissue hemodynamics, in addition to methods for positioning a health monitoring device on a user's body.

### SUMMARY

Embodiments according to the present invention pertain to sensor devices that accurately detect and report anomalies relating to cardiovascular activity or tissue hemodynamics, and also to methods for positioning a health monitoring device on a user's body. The sensor devices can be used in conjunction with computing systems (e.g., mobile systems) for providing real-time cardiovascular sensing.

In embodiments, a skin tissue sensor device includes a processor, a number of spaced-apart light-emitting diodes, and a number of photo-detectors matched by wavelength to the light-emitting diodes. The photo-detectors measure electromagnetic waves emitted by the light-emitting diodes after transport through an individual's skin tissue, and the processor is operable to record vital signs of the individual, based on measurement data provided by the skin tissue sensor device.

In embodiments, the vital signs include, for example, cardiovascular activity, tissue oxygenation and hemodynamics, and blood perfusion.

In embodiments, the processor detects anomalies that exist in the vital signs. In an embodiment, detected anomalies are analyzed by the processor to decide on an appropriate response. In an embodiment, the appropriate response is reported by the processor to a support system.

In embodiments, the skin tissue sensor device also includes at least one capacitive element and at least one metal pad that are configured for contact with the individual's skin. In an embodiment, the processor determines if the sensor device is being worn and is properly positioned, e.g., in proper contact with the individual's body, based on measurement data from the at least one capacitive element and the at least one metal pad.

In embodiments, the skin tissue sensor device also includes a temperature sensor coupled to the processor. In an embodiment, the temperature sensor provides temperature data to the processor, and the processor uses the temperature data to calibrate the skin tissue sensor.

In embodiments, the skin tissue sensor device also includes an accelerometer. In an embodiment, the accelerometer provides acceleration data to the processor, and the processor uses the acceleration data to infer user activity level. In an embodiment, the processor also uses the acceleration data to infer whether a physical mishap occurred to the individual.

In an embodiment, the spaced-apart light-emitting diodes include at least a first pair of light-emitting diodes that emit a visible-to-human wavelength and at least a second pair of light-emitting diodes that emit an infra-red wavelength.

In other embodiments, a symmetrical skin tissue sensor device includes a number of sensor elements, including: a first set of capacitive pads configured for skin contact; a second set of visible light-emitting diodes; a third set of infra-red light-emitting diodes; and a fourth set of photo-detecting diodes. Each of the first, second, third, and fourth sets includes an even number of members of the sensor elements, and there is symmetry between opposing members of each of the first, second, third, and fourth sets, in which the symmetry is equidistance (equidistant spacing) from a central x-axis of the opposing members and equidistance from a central y-axis of the opposing members. In an embodiment, the skin tissue sensor device also includes a number of metal pads configured for skin contact.

In other embodiments, an asymmetrical skin tissue sensor device includes: a number of sensing pads for inferring skin contact and correct positioning of the asymmetrical skin tissue sensor on a user's body; a number of visible light-emitting diodes; a number of infra-red light-emitting diodes; and a number of photo-detecting diodes. The photo-detecting diodes are matched by wavelength with the visible light-emitting diodes and with the infra-red light-emitting diodes. In an embodiment, a selected one of the visible light-emitting diodes and a matched corresponding one of the photo-detecting diodes form a matched pair, in which the distance between the matched pair is defined as a baseline, where multiple baselines are provided for the matched pair.

In embodiments, a method for sensing the vital signs of an individual with a digital monitoring device includes measuring reflections of light originally emitted from a number of spaced-apart light-emitting diodes, after the light has traversed skin tissue of the individual, using a number of spaced-apart photo-detectors, where the spaced-apart light-emitting diodes have multiple frequencies and the spaced-apart photo-detectors are matched by wavelength with the spaced-apart light-emitting diodes, and where the spaced-apart light-emitting diodes and the spaced-apart photo-detectors are positioned face-to-face with the individual's skin. The method further includes: recording the measured reflections as absorption spectra; analyzing the absorption spectra to determine the vital signs; detecting if any abnormalities exist in the vital signs; deciding on an appropriate response to report about the vital signs; and reporting the appropriate response to a support system.

In an embodiment, the method also includes calibrating the digital monitoring device using temperature data provided by a temperature sensor of the digital monitoring device.

In an embodiment, the method also includes determining if the digital monitoring device is properly positioned on the individual using data obtained from sensors adjacent the individual's skin configured for determining skin contact.

In an embodiment, the method also includes determining an activity level for the individual and determining if the individual has suffered a physical mishap, using data obtained from an accelerometer of the digital monitoring device.

In embodiments, a method for positioning a wearable health monitoring system on a user's body uses a number of skin sensing elements on a face on the wearable health monitoring system that is compatible with a portion of the user's anatomy, and also uses a detection circuit for each of the skin sensing elements, where the skin sensing elements are spaced apart, each element having at least a portion disposed adjacent a perimeter of the face. In these embodiments, the method includes: detecting a quality of skin contact using each of the detection circuits; and processing the quality of skin contacts to determine if the health monitoring system is properly positioned on the user.

These and other objects and advantages of the various embodiments according to the present invention will be recognized by those of ordinary skill in the art after reading the following detailed description of the embodiments that are illustrated in the various drawing figures.

### BRIEF DESCRIPTION OF THE DRAWINGS

The accompanying drawings, which are incorporated in and form a part of this specification and in which like numerals depict like elements, illustrate embodiments according to the present invention and, together with the detailed description, serve to explain the principles of the invention.

FIG. 1 is a cross-sectional view of a wrist-worn sensor device in an embodiment according to the present invention.

FIG. 2A is a plan view of a skin tissue sensor device having four capacitive sensing elements in an embodiment according to the present invention.

FIG. 2B is a plan view of a skin tissue sensor device having two capacitive sensing elements in an embodiment according to the present invention.

FIG. 2C is a plan view of a skin tissue sensor device having two capacitive sensing elements and two metal pads in an embodiment according to the present invention.

FIG. 2D is a plan view of a skin tissue sensor device having two metal pads in an embodiment according to the present invention.

FIG. 2E is a plan view of a skin tissue sensor device having four metal pads in an embodiment according to the present invention.

FIG. 3 is a schematic view of a detection circuit associated with a capacitive sensor element in an embodiment according to the present invention.

FIG. 4 is a schematic view of a detection circuit associated with a metal pad, in an embodiment according to the present invention.

FIG. 5 is a graph of sensor voltage versus time in an embodiment according to the present invention.

FIG. 6 is a plan view of a symmetrical sensor array in an embodiment according to the present invention.

FIG. 7 is a plan view of an asymmetric sensor array in an embodiment according to the present invention.

FIG. 8A is a flowchart of an example of a method for providing and using a skin tissue sensor device in a health monitoring system, or digital monitoring device, in an embodiment according to the present invention.

FIG. 8B is a flowchart of an example of a computer-implemented method for sensing the vital signs of an individual using a skin tissue sensor device, in an embodiment according to the present invention.

FIG. 9A is a flowchart of an example of a method for positioning a skin tissue sensor device in a health monitoring system on a user, in an embodiment according to the present invention.

FIG. 9B is a flowchart of an example of a computer-implemented method for correctly positioning a skin tissue sensor device on a user in an embodiment according to the present invention.

### DETAILED DESCRIPTION

Reference will now be made in detail to the various embodiments according to the present invention, examples of which are illustrated in the accompanying drawings. While described in conjunction with these embodiments, it will be understood that they are not intended to limit the invention to these embodiments. On the contrary, the disclosure is intended to cover alternatives, modifications and equivalents, which may be included within the spirit and scope of the disclosure as defined by the appended claims.

Furthermore, in the following detailed description of the present invention, numerous specific details are set forth in order to provide a thorough understanding of the present invention. However, it will be understood that the present invention may be practiced without these specific details. In other instances, well-known methods, procedures, components, and circuits have not been described in detail so as not to unnecessarily obscure aspects of the present invention.

Some portions of the detailed descriptions that follow are presented in terms of procedures, logic blocks, processing, and other symbolic representations of operations on data bits within a computer memory. These descriptions and representations are the means used by those skilled in the data processing arts to most effectively convey the substance of their work to others skilled in the art. In the present application, a procedure, logic block, process, or the like, is conceived to be a self-consistent sequence of steps or instructions leading to a desired result. The steps are those utilizing physical manipulations of physical quantities. Usually, although not necessarily, these quantities take the form of electrical or magnetic signals capable of being stored, transferred, combined, compared, and otherwise manipulated in a computing system. It has proven convenient at times, principally for reasons of common usage, to refer to these signals as transactions, bits, values, elements, symbols, characters, samples, pixels, or the like.

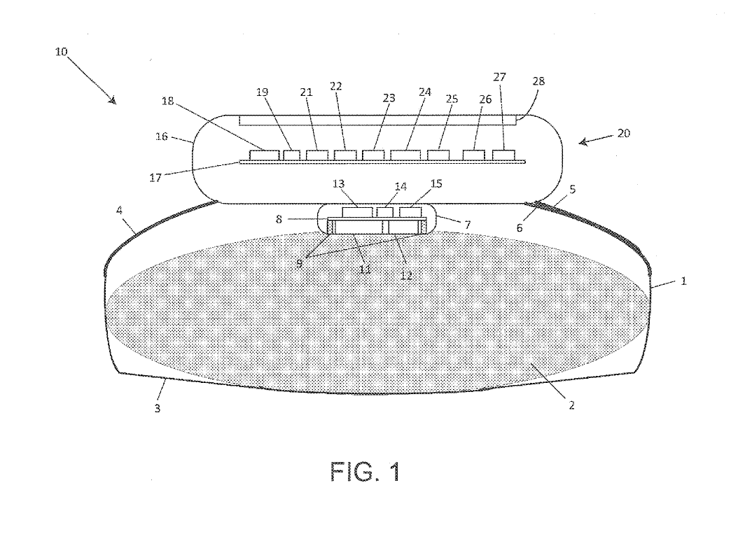

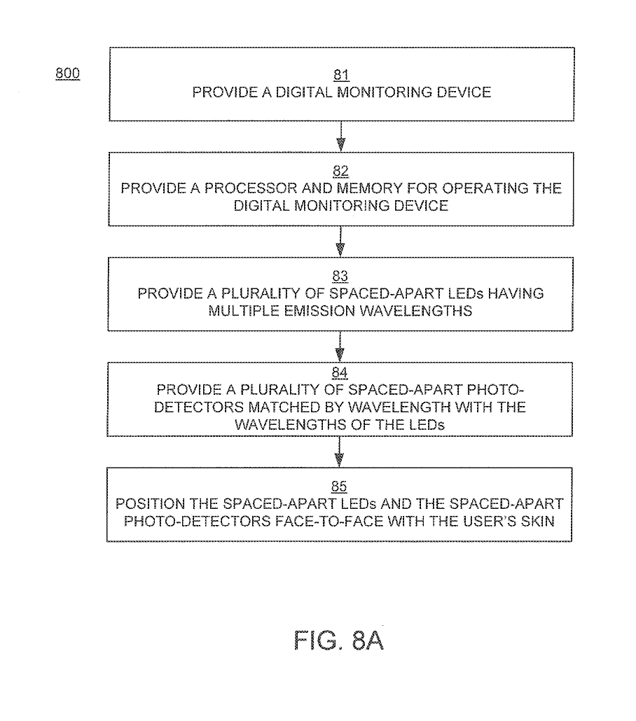

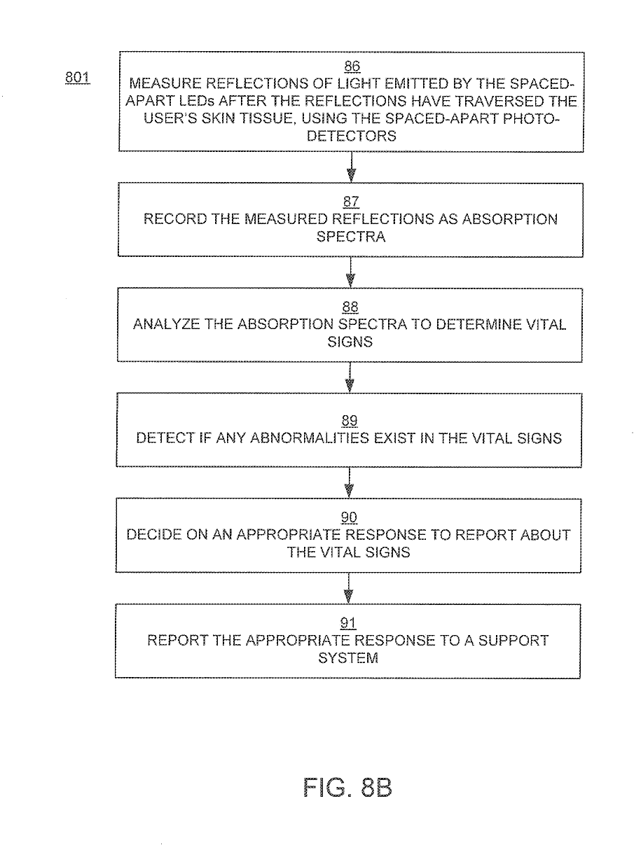

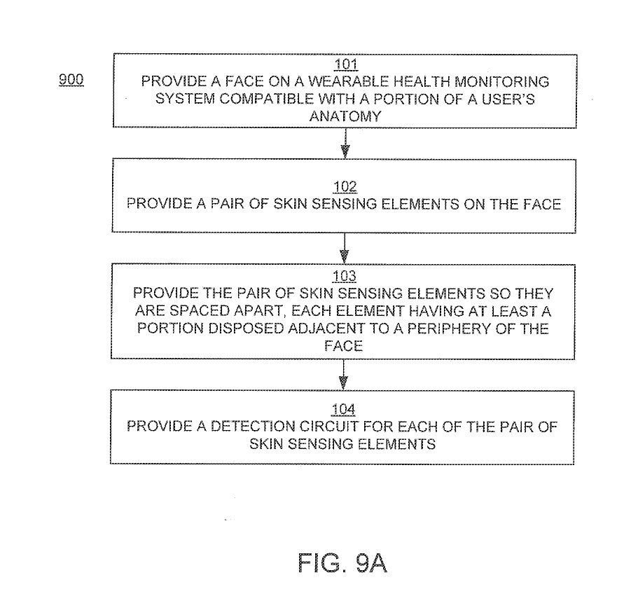

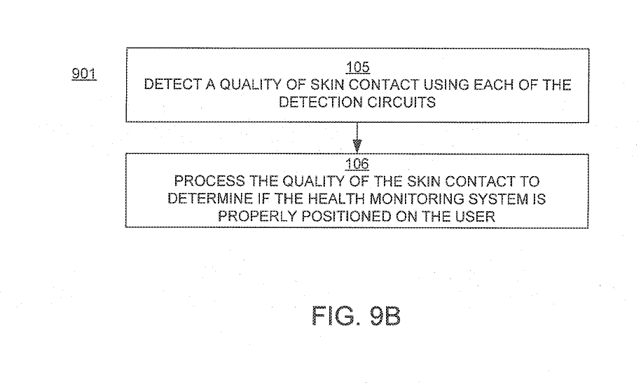

It should be borne in mind, however, that all of these and similar terms are to be associated with the appropriate physical quantities and are merely convenient labels applied to these quantities. Unless specifically stated otherwise as apparent from the following discussions, it is appreciated that throughout the present disclosure, discussions utilizing terms such as “measuring,” “detecting,” “analyzing,” “reporting,” “determining,” “providing,” “using,” “inferring,” “deciding,” “calibrating,” “processing,” or the like, refer to actions and processes (e.g., the flowcharts of FIGS. 8B and 9B) of a computing system or similar electronic computing device or processor (e.g., the health monitoring system 10 of FIG. 1). The computing system or similar electronic computing device manipulates and transforms data represented as physical (electronic) quantities within the computing system memories, registers or other such information storage, transmission or display devices.

Embodiments described herein may be discussed in the general context of computer-executable instructions residing on some form of computer-readable storage medium, such as program modules, executed by one or more computers or other devices. By way of example, and not limitation, computer-readable storage media may comprise non-transitory computer-readable storage media and communication media; non-transitory computer-readable media include all computer-readable media except for a transitory, propagating signal. Generally, program modules include routines, programs, objects, components, data structures, etc., that perform particular tasks or implement particular abstract data types. The functionality of the program modules may be combined or distributed as desired in various embodiments.

Computer storage media includes volatile and nonvolatile, removable and non-removable media implemented in any method or technology for storage of information such as computer-readable instructions, data structures, program modules or other data. Computer storage media includes, but is not limited to, random access memory (RAM), read only memory (ROM), electrically erasable programmable ROM (EEPROM), flash memory or other memory technology, compact disk ROM (CD-ROM), digital versatile disks (DVDs) or other optical storage, magnetic cassettes, magnetic tape, magnetic disk storage or other magnetic storage devices, or any other medium that can be used to store the desired information and that can accessed to retrieve that information.

FIG. 1 depicts a cross-sectional view of a wearable, wrist-mounted health monitoring system, or digital monitoring device, 10 that includes an example embodiment of a skin tissue sensor device 20 according to the present invention. The system 10 includes an attachment band 1 that fits over a user's wrist 2. A band 1 includes a stiffening element 3 that provides a desired level of pressure between the wrist 2 and the sensor device 20. A cellular antenna 4 supports cellular communications. A Global Navigation Satellite System (GNSS) antenna 5 is also provided. GNSS is a Global Positioning Satellite (GPS) system implemented in the United States, as well as in other nations. A diversity antenna 6 is additionally provided to augment the other antennas and improve their performance.

In FIG. 1, a sensor module 7 is shown, with its bottom surface pressing against the user's skin; this module incorporates the skin tissue sensor device 20. In the FIG. 1 embodiment, the module 7 includes a first printed circuit board 8, an array of conductive elements or capacitors 9 for confirming good contact with the user's wrist; an array of sensing circuits (not shown) associated with the array of conductive elements or capacitors for determining proper disposition of the skin tissue sensor device 20 relative to the user's body; light-emitting diodes 11 (LEDs) having multiple operating frequencies; photo-detectors 12 for measuring light originating from the LEDs 11 that subsequently diffuses through the user's blood and skin tissue; a temperature sensor 13 for recording ambient temperature and supporting calibration of the sensor device 20; an accelerometer 14 for sensing falls and accidents, and for inferring (determining) user activity or that a user has suffered a physical mishap; and a first microprocessor 15 for controlling the sensor module.

The light-emitting diodes 11 advantageously include a number of spaced-apart LEDs operating at multiple frequencies. The light-emitting diodes 11 and photo-detectors 12 are advantageously arranged in a predetermined array format, such that absorption spectra may be measured. The detection wavelength of each photo-detector is matched to the emission wavelength of a corresponding light-emitting diode; the photo-detectors are thus matched by wavelength to the light-emitting diodes.

Spatial spectrometry or spectroscopy is used to determine the absorption spectra. The absorption spectra are analyzed to determine vital signs of the user, and the vital signs may be used to make inferences about the user's health. With regard to cardiac activity, peaks and valleys in the absorption spectra sampled over time can be used to determine heart rate, for example.

The temperature sensor 13 is used to track the temperature of the sensor module 7 versus time; the temperature data collected is used to compensate for temperature drift in LED and photodiode characteristics. For example, the light output from an LED 11 typically varies with temperature, but this variation is nullified in real time by applying a compensation factor calculated from the current temperature.

FIG. 1 further illustrates an enclosure 16 surrounding a computer module that includes, for example, the following elements: a second printed circuit board 17; an embedded subscriber identification module, SIM 18; cellular module 19 for supporting cellular communications; a second microprocessor 21 for controlling the computer module; flash memory 22; SDRAM (synchronous dynamic random access memory) 23; battery and power management controller 24; a charging interface 25 for charging a battery (not shown); a Global Navigation Satellite System (GNSS) module 26; a voice chip 27; and a touch/display screen 28. The voice chip 27 is operable to create spoken text for signaling the user and potential local responders.

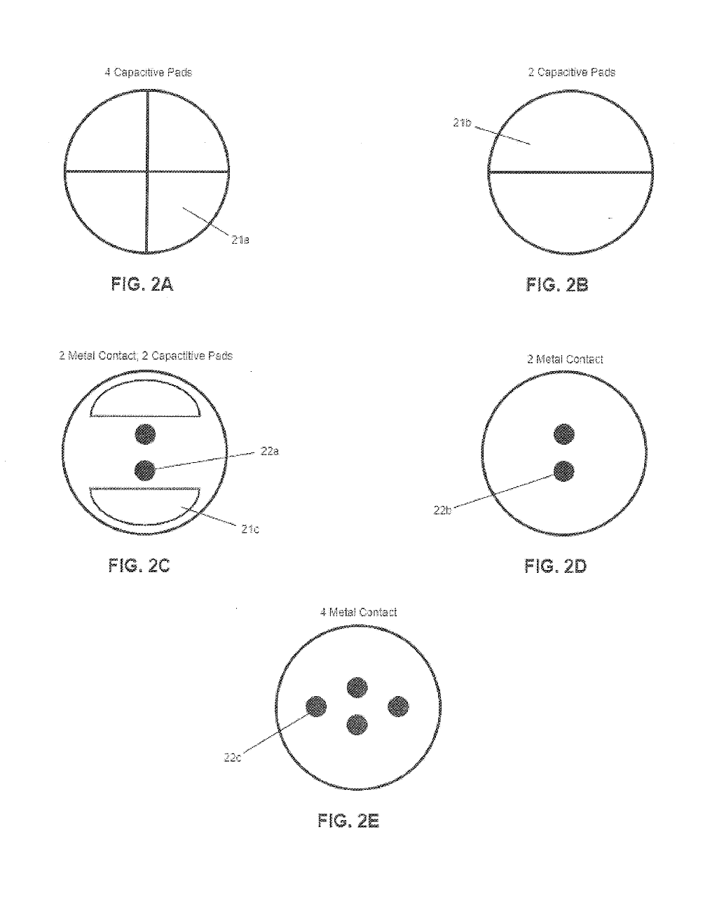

FIG. 2A shows an array of four capacitive pads 21a, used for detecting proper skin contact, in an embodiment according to the present invention. Each capacitive pad includes an insulating layer adjacent the user's skin, and a conductive layer coupled to the insulating layer.

FIG. 2B shows an array of two capacitive pads 21b, used for detecting proper skin contact, in an embodiment according to the present invention. Each capacitive pad includes an insulating layer adjacent the user's skin, and a conductive layer coupled to the insulating layer.

FIG. 2C depicts an array of elements (sensing pads) for detecting skin contact in an embodiment according to the present invention, including a pair of capacitive pads 21c and a pair of metal pads 22a, where each capacitive pad 21c includes an insulating layer adjacent the user's skin and a conductive layer coupled to the insulating layer, and each metal pad 22a includes a conductive layer in direct contact with a user's skin.

FIG. 2D shows a pair of metal pads 22b for detecting skin contact in an embodiment according to the present invention, where each metal pad 22b includes a conductive layer in direct contact with a user's skin.

FIG. 2E shows an array of four metal pads 22c for detecting skin contact in an embodiment according to the present invention, where each metal pad 22c includes a conductive layer in direct contact with a user's skin.

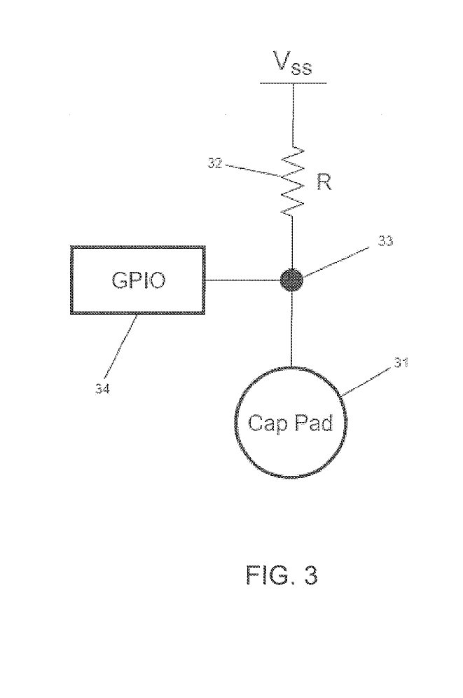

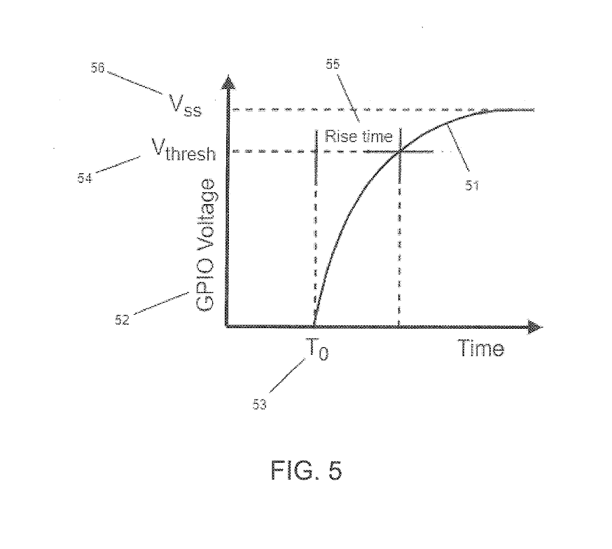

FIG. 3 illustrates an electronic detection circuit for receiving sensor data from a capacitive pad 31, in an embodiment according to the present invention. The capacitive pad 31 represents the capacitive pads such as the pads 21a, 21b, and 21c, described above. The capacitive pad 31 is connected through a resistor R 32 to a voltage supply VSS as shown, and the common node 33 is connected to a general-purpose input/output (GPIO) pin 34 of the first microprocessor 15 of the skin tissue sensor device 20. When the capacitive pad 31 is in contact with a user's skin, the user's capacitance will add in parallel to the capacitance value of the pad 31, providing a way to detect skin contact, to be further described with reference to FIG. 5.

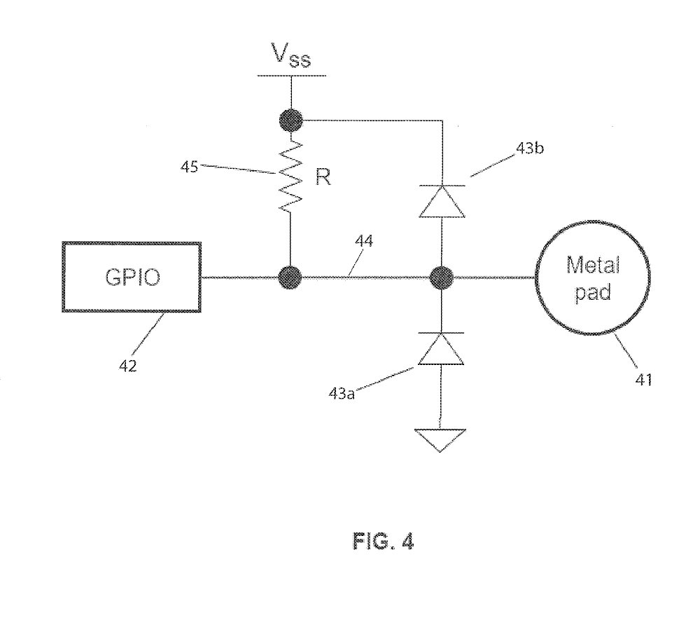

FIG. 4 depicts an electronic detection circuit for receiving sensor data from a metal pad 41, in an embodiment according to the present invention. The metal pad 41 represents the metal pads 22a, 22b, and 22c, described above. To protect the GPIO pin 42 from high voltage transients that may be present when the pin is first connected to a user's skin, diodes 43a and 43b are provided. The diodes 43a and 43b may be described as static protection diodes. A common node 44 connects the metal pad 41 to the GPIO pin 42, and a resistor R 45 is connected from a voltage supply VSS to the common node 44 as shown. Sensor data on the node 44 varies depending on whether or not the metal pad 41 is in contact with a user's skin, providing an additional means to verify that the sensor module 7 is in proper contact with a user's body.

FIG. 5 shows an example waveform 51 plotted as a GPIO input voltage 52 versus time. The waveform 51 is produced by charging a capacitive pad such as the pad 31 described with reference to FIG. 3. The capacitor is initially discharged to ground (VSS) through the GPIO. A positive voltage is applied at the GPIO pin at time T₀ 53, and a threshold 54 is sensed after rise-time 55.

The rise-time 55 depends on the total capacitance on the measurement node 33 of FIG. 3, and increases when a user's body capacitance is added in parallel to the capacitance of the pad 31. This increase in capacitance and the associated increase in rise-time can be used to reliably detect skin contact at a skin sensing element. By deploying a pair of capacitive skin sensing elements such as the pads 21b or 21c described with reference to FIGS. 2B and 2C, or by deploying an array of capacitive skin sensing elements such as the pad 21a in FIG. 2A, a rise-time such as the rise-time 55 can also be used to infer a quality and adequacy of the skin contact at each skin sensing element, and a plurality of rise-times like the rise-time 55 can be used to infer that the sensor device 20 is being worn by a user, and that the sensor device 20 is either properly or improperly positioned on the user's body.

Additional confidence about proper positioning of the sensor device 20 can be attained by measuring waveforms associated with nodes such as the node 44 of FIG. 4. This information is also employed by cardiac detection algorithms of the present invention, so that, for example, an emergency alert will not issue if there is insufficient confidence about good skin contact. If it is determined that the sensor device 20 is improperly positioned, a signal to the user will be provided, such as a vibration transmitted to the user's body and/or a displayed message on the touch/display screen. The user can then adjust the positioning of the module 7, and will be informed by a displayed message when proper positioning is achieved.

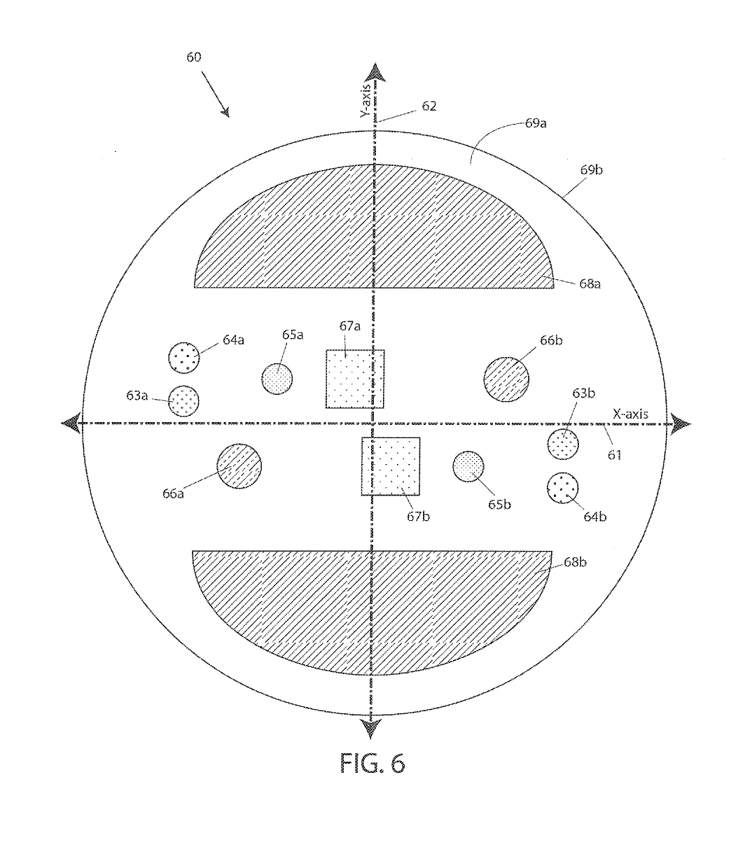

FIG. 6 illustrates a skin tissue sensor device 60 having symmetry with respect to the x-axis 61 and the y-axis 62, in an embodiment according to the present invention. Note, for example, that the sensor element 63a is equidistant from the x-axis 61 and the y-axis 62 with respect to the sensor element 63b, where the sensor elements 63a and 63b have similar properties (for example, an emission wavelength in the same range). The face of the skin tissue sensor 60 is shown as 69a and its perimeter as 69b.

The following pairs of skin sensing elements have similar symmetry and similar properties: the sensor elements 64a and 64b, 65a and 65b, 66a and 66b, 67a and 67b, and 68a and 68b. An example allocation of the sensor elements is: the sensor elements 63a and 63b are infra-red emitters; 64a and 64b are red emitters; 65a and 65b are green emitters; 66a and 66b are metal pads for sensing skin contact; 67a and 67b are photo-detectors having sensitivity for detecting wavelengths corresponding to the sensor elements 63a and 63b, 64a and 64b, 65a and 65b; and 68a and 68b are capacitive pads for sensing skin contact. Either or both of the metal pad pair 66a, 66b, and the capacitive pad pair 68a, 68b may be provided for sensing skin contact and correct positioning of the skin tissue sensor device 60 on a user's body. It is desirable that at least a portion of the skin sensing elements 68a and 68b, configured as capacitive elements in this example, is disposed adjacent the perimeter 69b of the face 69a.

During operation, electromagnetic waves emitted by sensor elements such as 63a, 63b; 64a, 64b; 65a, and 65b travel through skin tissue of the user, are absorbed and attenuated to a greater or lesser degree depending on the makeup of the user's blood and tissue and whether or not a heartbeat is in progress, and are subsequently received by the photo-detectors 67a and 67b. The received signals at the photo-detectors 67a and 67b are recorded as absorption spectra, and the recorded absorption spectra are analyzed by the skin tissue sensor 60 to determine vital signs. The vital signs are examined to determine if any anomalies are present. If anomalies are present, an appropriate response can be selected from, for example, a table of possible responses, and the response can be sent to the user and also sent to other parties (a support system) using cellular communications. The other parties may include health responders or a support network. Health responders may include, for example, doctors, nurses, or persons having defibrillators and the skill to use them. The support network may be a Public Safety Answering Point, PSAP. An example of a PSAP is a 911 call center.

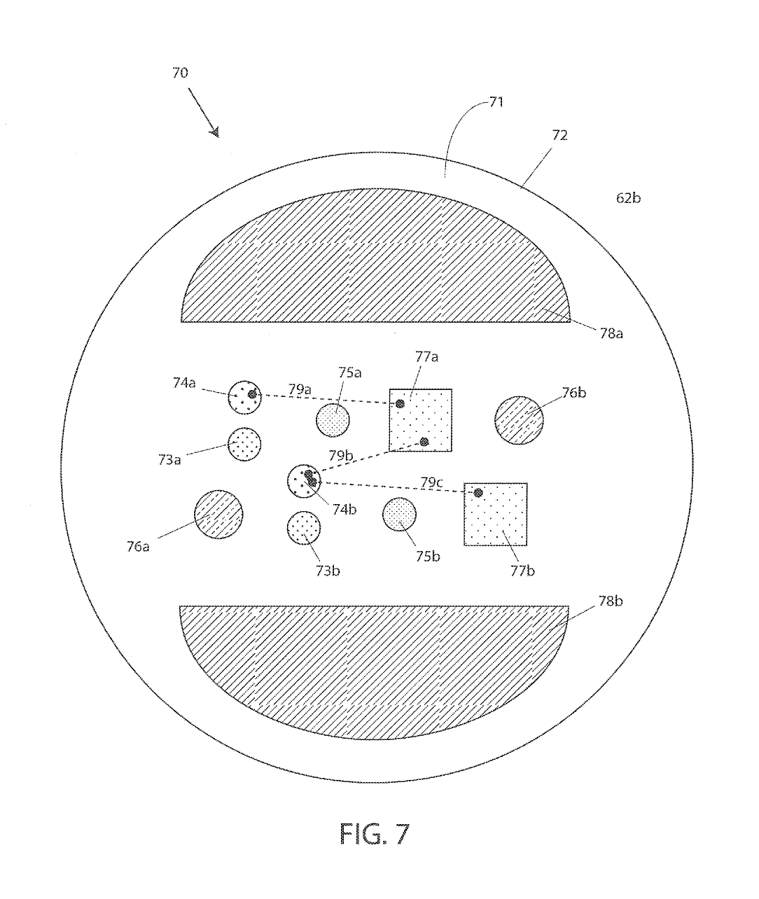

FIG. 7 depicts a skin tissue sensor device 70 that does not require symmetry of the sensing elements, in an embodiment according to the present invention. The face 71 and the perimeter 72 of the skin tissue sensor device 70 are shown. Sensing elements of the skin tissue sensor 70 are provided in pairs in this example. As an example allocation of sensing elements, 73a and 73b are infra-red emitting diodes; 74a and 74b are red emitting diodes; 75a and 75b are green emitting diodes; 76a and 76b are metal pads for detecting skin contact; 77a and 77b are photo-detecting diodes having sensitivity for detecting wavelengths corresponding to the sensing elements 73,73b, 74a, 74b, and 75a, 75b in this example; and 78a, 78b are capacitive elements for sensing skin contact and correct positioning of the skin tissue sensor device 70 on a user's body.

During operation, electromagnetic waves emitted by the sensor elements such as 73a, 73b; 74a, 74b; 75a, 75b travel through skin tissue of the user, are absorbed and attenuated to a greater or lesser degree depending on the makeup of the user's blood and tissue and whether or not a heartbeat is in progress, and are subsequently received by the photo-detectors 77a and 77b. The received signals at the photo-detectors 77a and 77b are recorded as absorption spectra, and the recorded absorption spectra are analyzed by skin the tissue sensor device 70 to determine vital signs. The vital signs are examined to determine if any anomalies are present. If anomalies are present, an appropriate response can be selected from, for example, a table of possible responses, and the response can be sent to the user and also sent to other parties (a support system) using cellular communications. The other parties may include health responders or a support network. Health responders may include, for example, doctors, nurses, or persons having defibrillators and the skill to use them. The support network may be a PSAP. An example of a PSAP is a 911 call center.

In FIG. 7, a baseline 79a is shown, and is defined as the distance between an emitting diode, 74a in this case, and a photo-detector matched by wavelength, 77a in this case. At the detection wavelength of the emitting diode 74a, alternative baselines are provided at the baselines 79b and 79c. Thus, a number of baselines is provided for each matched pair of emitter and photo-detector. The baselines are used to reduce measurement error by accounting for subtler variables such as orientation bias that may be otherwise unaccounted for.

Skin tissue measurements involve using a skin tissue sensor device, such as the sensor device 60 described in reference to FIG. 6 or the sensor device 70 described in reference to FIG. 7, to perform spatially resolved spectroscopy, which may be described as Near Infrared Spectroscopy, NIRS. Sensed dermal activity may include tissue oxygenation or hemodynamics measurements or blood perfusion measurements. From these measurements, inferences about a user's cardiovascular and hemodynamic activity can be made. Sensed cardiovascular activity includes, for example, heart rhythm monitoring and detection of anomalies such as hemorrhaging, fibrillation, or unusual cardiac rhythms such as asystole, atrial fibrillation, ventricular tachycardia, ventricular fibrillation, and bradycardia. Perfusion is defined herein as the passage of fluid through the circulatory system or lymphatic system to an organ or a tissue, and more particularly to the delivery of blood to a capillary bed in tissue; in embodiments according to the present invention, it is sensed by measuring the blood concentration in a user's skin tissue.

FIG. 8A is a flowchart of an example of a method 800 for providing and using a skin tissue sensor device in a health monitoring system, or digital monitoring device, in an embodiment according to the present invention.

The method 800 begins by providing the health monitoring system device (block 81).

The method 800 further includes providing a processor and memory for operating the digital monitoring device (block 82).

The method 800 further includes providing a plurality of spaced-apart LEDs having multiple emission wavelengths (block 83).

The method 800 further includes providing a plurality of spaced-apart photo-detectors having sensitivity matched by wavelength with the multiple emission wavelengths of the LEDs (block 84).

The method 800 further includes positioning the spaced-apart LEDs and the spaced-apart photo-detectors face-to-face with the individual's skin (block 85).

FIG. 8B is a flowchart of an example of a computer-implemented method 801 for sensing the vital signs of an individual using a skin tissue sensor device, in an embodiment according to the present invention.

The method 801 includes measuring reflections of light emitted by the spaced-apart LEDs after the reflections have traversed skin tissue of the individual, using the spaced-apart photo-detectors (block 86).

The method 801 further includes recording, into computer memory, the measured reflections as absorption spectra (block 87).

The method 801 further includes using a processor to analyze the absorption spectra to determine the vital signs (block 88).

The method 801 further includes using the processor to detect if any abnormalities exist in the vital signs (block 89).

The method 801 further includes using the processor to decide on (determine) an appropriate response to report about the vital signs (block 90).

The method 801 further includes sensing the vital signs of an individual by reporting the appropriate response to a support system (block 91).

FIG. 9A is a flowchart of an example of a method 900 for positioning a skin tissue sensor device in a health monitoring system on a user, in an embodiment according to the present invention.

The method 900 begins by providing a face on a wearable health monitoring system compatible with a portion of the user's anatomy (block 101).

The method 900 further includes providing a pair of skin sensing elements on the face of the wearable health monitoring system (block 102).

The method 900 further includes: the pair of skin sensing elements is spaced apart, each element having at least a portion disposed adjacent a periphery of the face (block 103).

The method 900 further includes providing a detection circuit for each of the pairs of skin sensing elements (block 104).

FIG. 9B is a flowchart of an example of a computer-implemented method 901 for correctly positioning a skin tissue sensor device on a user in an embodiment according to the present invention.

The method 901 includes detecting a quality of skin contact using each of the detection circuits (block 105).

The method 901 further includes properly positioning a wearable health monitoring system on a user's body by processing the quality of skin contacts to determine if the health monitoring system is properly positioned on the user (block 106). As described in conjunction with FIG. 3, a rise-time such as the rise-time 55 can also be used to infer a quality and adequacy of the skin contact at each skin sensing element, and a plurality of rise-times like the rise-time 55 can be used to infer that the sensor device is being worn by a user, and that the sensor device 20 is either properly or improperly positioned on the user's body.

The process parameters and sequence of steps described and/or illustrated herein are given by way of example only and can be varied as desired. For example, while the steps illustrated and/or described herein may be shown or discussed in a particular order, these steps do not necessarily need to be performed in the order illustrated or discussed. The example methods described and/or illustrated herein may also omit one or more of the steps described or illustrated herein or include additional steps in addition to those disclosed.

While the foregoing disclosure sets forth various embodiments using specific block diagrams, flowcharts, and examples, each block diagram component, flowchart step, operation, and/or component described and/or illustrated herein may be implemented, individually and/or collectively, using a wide range of hardware, software, or firmware (or any combination thereof) configurations. In addition, any disclosure of components contained within other components should be considered as examples because many other architectures can be implemented to achieve the same functionality.

Although the subject matter has been described in language specific to structural features and/or methodological acts, it is to be understood that the subject matter defined in the disclosure is not necessarily limited to the specific features or acts described above. Rather, the specific features and acts described above are disclosed as example forms of implementing the present invention.

Embodiments according to the invention are thus described. While the present invention has been described in particular embodiments, it should be appreciated that the invention should not be construed as limited by such embodiments, but rather construed according to the following claims.

What is claimed is:

1. A skin tissue sensor device, comprising:
a processor;
a memory coupled to the processor and operable for storing instructions executable by the processor;
a plurality of spaced-apart light-emitting diodes coupled to the processor; and
a plurality of photo-detectors coupled to the light-emitting diodes and matched by wavelength to the plurality of light-emitting diodes;
wherein the photo-detectors measure electromagnetic waves emitted by the light-emitting diodes after transport through skin tissue, and wherein the processor is operable to record vital signs, based on measurement data provided by the skin tissue sensor device.

2. The skin tissue sensor device of claim 1, wherein the vital signs are selected from the group consisting of: cardiovascular activity; tissue hemodynamics; and blood perfusion.

3. The skin tissue sensor device of claim 1, wherein the processor is further operable to detect anomalies in the vital signs.

4. The skin tissue sensor device of claim 3, wherein the processor is further operable to analyze the anomalies and decide on an appropriate response.

5. The skin tissue sensor device of claim 4, wherein the processor is further operable to report the appropriate response to a support system.

6. The skin tissue sensor device of claim 1, further comprising at least one capacitive element and at least one metal pad that are coupled to the processor and that are configured for contact with the skin tissue.

7. The skin tissue sensor device of claim 6, wherein the processor is further operable to determine if the sensor is being worn and in proper contact with the skin tissue based on data measured by the at least one capacitive element and the at least one metal pad.

8. The skin tissue sensor device of claim 1, further comprising a temperature sensor coupled to the processor.

9. The skin tissue sensor device of claim 8, wherein the temperature sensor is operable for providing temperature data to the processor, and wherein the processor is operable for using the temperature data to calibrate the skin tissue sensor.

10. The skin tissue sensor device of claim 1, further comprising an accelerometer coupled to the processor.

11. The skin tissue sensor device of claim 10, wherein the accelerometer is operable for providing acceleration data to the processor, and wherein the processor is operable for using the acceleration data to infer user activity level and is also operable for using the acceleration data to infer whether a physical mishap occurred to a user.

12. The skin tissue sensor device of claim 1, wherein the plurality of spaced-apart light-emitting diodes comprises at least a first pair of light-emitting diodes that emit a human-visible wavelength and at least a second pair of light-emitting diodes that emit an infra-red wavelength.

13. A skin tissue sensor device, comprising:
a plurality of sensor elements, comprising:
a first plurality of capacitive pads configured for skin contact;
a second plurality of visible light-emitting diodes;
a third plurality of infra-red light-emitting diodes; and
a fourth plurality of photo-detecting diodes;
wherein each of the first, second, third, and fourth pluralities comprises an even number of members of the plurality of sensor elements, and wherein there is symmetry between opposing members of each of the first, second, third, and fourth pluralities, the symmetry comprising equidistance from a central x-axis of the opposing members and equidistance from a central y-axis of the opposing members.

14. The skin tissue sensor device of claim 13, further comprising a plurality of metal pads configured for skin contact.

15. A skin tissue sensor device, comprising:
a plurality of sensing pads for detecting skin contact and correct positioning of the skin tissue sensor device on a user's body;
a plurality of visible light-emitting diodes coupled to the plurality of sensing pads;
a plurality of infra-red light-emitting diodes coupled to the plurality of sensing pads; and
a plurality of photo-detecting diodes coupled to the plurality of sensing pads;
wherein the plurality of photo-detecting diodes is matched by wavelength with the plurality of visible light-emitting diodes and with the plurality of infra-red light-emitting diodes.

16. The skin tissue sensor device of claim 15, wherein a selected light-emitting diode of the plurality of visible light-emitting diodes and a matched corresponding photo-detecting diode of the plurality of photo-detecting diodes form a matched pair, wherein the distance between the matched pair is defined as a baseline, and wherein a plurality of baselines is provided for the matched pair.

17. A computer-implemented method for sensing vital signs of an individual with a digital monitoring device comprising a processor and memory, the method comprising:
measuring reflections of light originally emitted from a plurality of spaced-apart light-emitting diodes, from light that has traversed skin tissue of the individual, using a plurality of spaced-apart photo-detectors, wherein the plurality of spaced-apart light-emitting diodes have multiple frequencies, wherein the plurality of spaced-apart photo-detectors are matched by wavelength with the plurality of spaced-apart light-emitting diodes, and wherein the spaced-apart light-emitting diodes and the spaced-apart photo-detectors are positioned face-to-face with the individual's skin tissue;
recording the reflections from said measuring as absorption spectra;
analyzing the absorption spectra to determine the vital signs;
detecting if any abnormalities exist in the vital signs;
determining an appropriate response regarding results of said detecting; and
reporting the appropriate response to a support system.

18. The method of claim 17, wherein the vital signs are selected from the group consisting of: cardiovascular data; tissue hemodynamics data; and blood profusion data.

19. The method of claim 17, further comprising calibrating the digital monitoring device using temperature data provided by a temperature sensor of the digital monitoring device.

20. The method of claim 17, further comprising determining if the digital monitoring device is properly positioned on the individual using data obtained from a plurality of sensor pads adjacent the individual's skin and configured for detecting skin contact.

21. The method of claim 17, further comprising determining an activity level for the individual and determining if the individual has suffered a physical mishap, using data obtained from an accelerometer of the digital monitoring device.

22. A method for positioning a sensor device on a user's body, the method comprising:
with a plurality of skin sensing elements on a face on the wearable health monitoring system that is compatible with a portion of the user's anatomy, and with a plurality of detection circuits comprising a detection circuit for each sensing skin element of the plurality of skin sensing elements, wherein the plurality of skin sensing elements are spaced apart, each skin sensing element of the plurality of skin sensing elements having at least a portion disposed adjacent a perimeter of the face:
detecting a quality of skin contact using each detection circuit of the plurality of detection circuits; and
processing the quality of skin contact detected by each detection circuit to determine if the health monitoring system is properly positioned on the user.
